(19) United States
(12) Patent Application Publication
(10) Pub. No.: US 2022/0062170 A1
(43) Pub. Date: Mar. 3, 2022

(54) INHALABLE DOSAGE FORM OF CANNABINOID EXTRACT

(71) Applicant: Columbia Care LLC, New York, NY (US)

(72) Inventors: Aaron Michael Dely, Lighthouse Point, FL (US); Shaun Ben-Ari, Rochester, NY (US)

(21) Appl. No.: 17/421,168

(22) PCT Filed: Jan. 7, 2020
(86) PCT No.: PCT/US20/12576

§ 371 Date: Jul. 7, 2021

Related U.S. Application Data
(60) Provisional application No. 62/789,417, filed on Jan. 7, 2019.

(51) Int. Cl. A61K 9/00; A61K 31/05; A61K 31/352; A61K 36/185; A61K 47/44; A61M 15/00

(52) U.S. Cl. CPC A61K 9/0073; A61K 31/05; A61M 15/0028; A61K 36/185; A61K 47/44; A61K 31/352

(57) ABSTRACT

Disclosed are cannabinoid formulations and methods of use associated therewith. Specifically, disclosed are inhalable dosage forms containing a solid, inert medium and a cannabinoid or cannabinoid extract that facilitates accurate and consistent dosing.

### RELATED APPLICATIONS

This application claims the benefit of priority to U.S. Provisional Application No. 62/789,417, filed Jan. 7, 2019, the contents of which are incorporated herein by reference in their entirety.

### FIELD OF INVENTION

Disclosed are cannabinoid formulations and methods of use associated therewith. Specifically, disclosed are inhalable dosage forms containing a solid, inert medium and a cannabinoid or cannabinoid extract that facilitates accurate and consistent dosing.

### BACKGROUND OF THE INVENTION

The medicinal and psychoactive properties of the cannabis plant have been known for centuries. While it has been illegal in many countries, there is a growing population lobbying for legalization of its use, especially for medicinal purposes.

Cannabis is believed to provide benefits in the treatment of multiple disorders with safer and fewer serious side effects than most prescription drugs currently used as antiemetics, muscle relaxants, hypnotics, and analgesics. A disadvantage in treating patients with cannabis is the psychoactive effect, especially in “naive” cannabis users. Furthermore, there have been reports of unpleasant reactions to cannabis, such as anxiety, panic, or hallucinations. It is believed that the undesirable side effects are most commonly associated with higher doses of cannabis and are related to the difficulty in controlling the dosage when the drug is smoked or eaten in cannabis-enriched confectionaries.

Cannabis has also been used to treat the symptoms in patients suffering from serious medical conditions. For example, cannabis has been used to alleviate symptoms associated with cancer, anorexia, AIDS, chronic pain, muscle spasticity, glaucoma, arthritis, migraine, and many other illnesses. Cannabis is recognized as having antiemetic properties and has been successfully used to treat nausea and vomiting in cancer patients undergoing chemotherapy. Cannabis has also been used in treating the weight loss syndrome of AIDS and in treating glaucoma by reducing intraocular pressure. Cannabis is also known for its muscle relaxing and anti-convulsant effects.

The most prevalent mode of administration of medical cannabis is by smoking. This mode of administration can have adverse effects on the lungs. Cannabis smoke carries more tar and other particulate matter than tobacco and may be a cause of lung diseases including lung cancer. Furthermore, many patients find the act of smoking unappealing, as well as generally unhealthy.

Cannabinoids and other active agents also can be administered in liquid compositions formulated for personal vaporizers—devices that typically utilize a small battery-powered atomizer or heater to turn a liquid into a vapor so that a subject can inhale the vapor. Many personal vaporizers are relatively small and inherently portable and come in a variety of forms.

Typically, personal vaporizers contain a liquid solution. The basic components of this liquid solution are: cannabinoid or cannabinoid extract, propylene glycol and/or vegetable glycerine base, and optionally a flavor. While some of the ingredients listed above are known to be generally safe in food and drugs, what remains unclear are the health risks that come from inhaling large amounts of the chemicals over time. Long-term effects are almost guaranteed.

There remains a need in the art, therefore, for an improved, inhalable dosage form for the accurately measured administration of one or more cannabinoids without the addition of any potentially harmful excipients or solvents.

### SUMMARY OF THE INVENTION

The present invention provides a single-use method of administering a cannabinoid-containing formulation via inhalation of vapor in an accurate and repeatable manner. The design of the formulations and devices disclosed herein permit users to easily store and carry multiple accurately quantified doses.

Traditional cartridge-based vaporization methods are vulnerable to inaccurate administration due to several variables including, but not limited to, power management, airflow, and viscosity. The dosage forms of the present invention are tailored to create a uniform aerosol for inhalation, reducing the risk of inaccurate dosing.

The structure of the solid, inert medium that acts as the vehicle for carrying an isolated cannabis extract permits complete vaporization of the cannabinoid formulation, preferably without any structural change to the medium, thus allowing for safe disposal of the medium post-use. In addition, the present invention avoids the use of potentially harmful ingredients that are common components of liquid solutions typically used in personal vaporizers.

In one aspect, provided herein are dosage forms suitable for use in an inhalation device, comprising a solid, inert medium containing a cannabinoid or cannabinoid extract. In certain embodiments, the cannabinoid or cannabinoid extract is absorbed onto the solid medium, e.g., a core. In certain embodiments, said medium comprises organic and inorganic compounds.

In certain embodiments, the dosage form provided herein further comprises a pharmaceutically acceptable solvent (e.g., selected from vegetable oil, hydrogenated vegetable oils, limonene, a terpene, an essential oil, polyethylene glycols (PEG), propylene glycol, ethanol, substituted polyethylene glycols, glycerin, mineral oil, oleic acid, fatty acid esters, benzyl alcohol, an alcohol, or any combinations thereof, preferably selected from hydrogenated vegetable oils, ethanol, or a mixture thereof) into which the cannabinoid is solvated and a pharmaceutically acceptable adsorbent (e.g., silica, microcrystalline cellulose, cellulose, silicified microcrystalline cellulose, starch, pregelatinized starch, dicalcium phosphate, or a mixture thereof, preferably silica) onto which the solvated cannabinoid is adsorbed.

In certain embodiments, the dosage form provided herein further comprises one or more other pharmaceutically acceptable excipients, which may be selected from diluents, lubricants, granulating aids, colorants, flavorants, surfactants, pH adjusters, anti-adherents, glidants, and any combination thereof.

In certain embodiments, said lubricants are tableting lubricants, e.g., selected from magnesium stearate, stearic acid, palmitic acid, calcium stearate, talc, polyethylene glycol, colloidal silicon dioxide, sodium stearyl fumarate, carnauba wax, and mixtures thereof. The tableting lubricants may be present in an amount of from about 0.2% to about 8% by weight, e.g., from about 0.5% to about 2% by weight.

Suitable flavorants include natural and synthetic flavorants, e.g., vanilla, strawberry, cherry, grape, lemon, lime, orange, peppermint, spearmint, cinnamon, or any combination thereof. The flavorants may be present in an amount of from about 0.005% to about 20% by weight, e.g., from about 0.01% to about 5% by weight.

The dosage form may be a pellet. The dosage form may be temperature dependent and/or capable of accurate dosing in an inhalation device.

The dosage form may be for single use, e.g., for daily administration to a human subject. Therapeutic effect may be maintained with one administration, twice daily. The duration of the therapeutic effect of a single dose may be between four and six hours.

The inhalation device may be a personal vaporizer. The cannabinoid or cannabinoid extract in the dosage form may be for administration in a vaporized form.

The dosage form may be sized and shaped to be inserted into the vaporization chamber of the inhalation device. Alternatively or additionally, the dosage form may be configured for insertion into a chamber, e.g., a vaporization chamber, having a shape that has sufficient surface area for complete vaporization.

The dosage form may be in the form of a disc or cylinder, and/or in a single, measured unit.

In certain embodiments, the cannabinoid or canninboid extract is selected from tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), and cannabichromene (CBC). In certain embodiments, the cannabinoid or cannabinoid extract comprises at least two cannabinoids, which may be selected from tetrahydrocannabinol (THC), cannabidiol (CBD), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), and derivatives thereof. Preferably, the two cannabinoids are THC and CBD or THCa and CBDa.

In certain embodiments, the two cannabinoids are in a 1:1 proportion by weight, in a 10:1 proportion by weight, or in a 20:1 proportion by weight.

In certain embodiments, the total amount of the cannabinoid or cannabinoid extract is between about 0.1 mg and about 10 mg. For example, the cannabinoid or cannabinoid extract may be THC present in an amount ranging from about 0.1 mg to about 10 mg, or the cannabinoid or cannabinoid extract may be CBD present in an amount ranging from about 0.1 mg to about 10 mg.

In another aspect, provided are methods for treating a disease or a disorder in a subject, the methods comprising administering, for example, inhalable dosage forms disclosed herein. Also provided herein are methods for treating a disease or a disorder in a subject, comprising administering to the subject a therapeutically effective amount of the dosage form disclosed herein.

The disease or the disorder may be selected from pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis, and dystonia.

Other features and advantages of the present invention will become apparent from the following detailed description and examples. It should be understood, however, that the detailed description and the specific examples, while indicating embodiments of the invention, are given by way of illustration only, since various changes and modifications within the spirit and scope of the invention will become apparent to those skilled in the art from this detailed description.

### DETAILED DESCRIPTION OF THE INVENTION

Before the present invention is disclosed and described, it is to be understood that this invention is not limited to the particular structures, process steps, or materials disclosed herein, but is extended to equivalents thereof as would be recognized by those of ordinary skill in the relevant arts. It should also be understood that the terminology employed herein is used for the purpose of describing particular embodiments only and is not intended to be limiting.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat,” “treatment,” or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” “medicament,” or “food product” are used interchangeably herein, as context dictates, to refer to a compound or compounds or composition of matter which, when administered to a subject (human or animal), induces a desired pharmacological and/or physiologic effect by local and/or systemic action. In certain embodiments, a dosage form is a composition.

The terms “subject,” “individual,” and “patient” are used interchangeably herein, and refer to an animal, for example a human, to whom treatment with a composition or formulation or food product in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dogs, rodents, (e.g., mice or rats), guinea pigs, goats, pigs, cats, rabbits, cows, horses, and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In certain embodiments, the mammal to be treated is human. The human can be any human of any age. In certain embodiments, the human is an adult. In certain embodiments, the human can be male, female, middle-aged, adolescent, or elderly. According to any of the methods of the present invention and in certain embodiments, the subject is human.

Conditions and disorders in a subject for which a particular drug, compound, composition, formulation, food product, or dietary supplement (or combination thereof) is said herein to be “indicated” are not restricted to conditions and disorders for which that drug or compound or composition or formulation or food product or dietary supplement has been expressly approved by a regulatory authority, but also include other conditions and disorders known or reasonably believed by a physician or other health or nutritional practitioner to be amenable to treatment with that drug or compound or composition or formulation or food product or dietary supplement or combination thereof

Standard conditions for cannabinoid assays and methods of calculating cannabinoid content (as %) are well known in the art.

In one aspect, provided are dosage forms suitable for use in an inhalation device, comprising a solid, inert medium containing a cannabinoid or cannabinoid extract.

In certain embodiments, the cannabinoid or cannabinoid extract is absorbed onto the solid medium. In certain embodiments, said medium is a core.

In certain embodiments, the composition is a solid, inert medium containing a cannabinoid or cannabinoid extract. In other embodiments, the composition is a solid, inert core made up of organic and inorganic compounds (e.g., ceramics) which act as a vehicle in the administration of a cannabinoid containing formulation.

In some embodiments, the solid, inert core is in the form of a tablet or pellet which is intended to be vaporized in a suitable vaporization device. In other embodiments, the solid, inert core is in a dosage form and contains a cannabis extract in an isolated form, capable of accurate dosing as an inhaled vapor.

In some embodiments, the dosage form is capable of accurate dosing via an inhalation device. In certain embodiments, “accurate dosing” means that the amount of cannabinoid ingested by the subject can be programmed and measured. For a typical vaporizer, the amount of active ingredient taken in depends on how deep a breath is the subject takes. Therefore, it is desirable to administer the same dose despite how deep or shallow a given breath is. Inhalation devices suitable for use with the dosage forms of the present disclosure include metered dose devices and regulated pumps. In certain embodiments, the inhalation device comprises a regulated pump. In certain embodiments, the device comprises a metered-dosing mechanism. In certain embodiments, the device comprises an air-tight container to store the formulation. In certain embodiments, the device comprises the formulation in a plurality of metered doses. In certain embodiments, the device is an aerosol spray device.

In other embodiments, the solid, inert core contains an isolated cannabis extract and allows for single use.

In certain embodiments, the dose, in the form of a solid, inert core containing an isolated cannabis extract, has the requisite structural strength to maintain its form either during manufacture or packaging over its shelf life.

In certain embodiments, the composition comprises: a cannabinoid; a pharmaceutically acceptable solvent into which the cannabinoid is solvated; and a pharmaceutically acceptable adsorbent onto which the solvated cannabinoid is adsorbed.

In some embodiments, the solvent comprises a vegetable oil, hydrogenated vegetable oils, limonene, a terpene, an essential oil, polyethylene glycols (PEG), propylene glycol, ethanol, substituted polyethylene glycols, glycerin, mineral oil, oleic acid, fatty acid esters, benzyl alcohol, an alcohol, or combinations thereof. In certain embodiments, the solvent comprises hydrogenated vegetable oils, ethanol, or mixtures thereof.

In some embodiments, the adsorbent comprises silica, microcrystalline cellulose, cellulose, silicified microcrystalline cellulose, starch, pregelatinized starch, dicalcium phosphate, or mixtures thereof. In other embodiments, the adsorbent comprises silica.

In certain embodiments, the composition comprises other pharmaceutically acceptable excipients, incorporated to ease the manufacturing process as well as to improve the performance of the dosage form. In other embodiments, excipients include diluents, lubricants, granulating aids, colorants, flavorants, surfactants, pH adjusters, anti-adherents, glidants, or combinations thereof. In other embodiments, excipients are incorporated in the dosage forms of this invention.

In certain embodiments, the inhalable solid oral dosage forms are in the form of tablets, capsules, pellets, granules, powders, coated granules, or coated pellets. In other embodiments, the dosage form is a tablet. In other embodiments, the dosage form is a pellet.

In certain embodiments, the composition is in the form of a tablet and includes one or more tableting lubricants. In other embodiments, the tableting lubricants are in an amount within the range of from about 0.2% to about 8%. In other embodiments, the tableting lubricants are in an amount within the range of from about 0.5% to about 2% by weight of the composition. In other embodiments, the tableting lubricant is magnesium stearate, stearic acid, palmitic acid, calcium stearate, talc, polyethylene glycol, colloidal silicon dioxide, sodium stearyl fumarate, carnauba wax, or the like, or mixtures thereof

In certain embodiments, flavorants or flavors are used to enhance the organoleptic qualities of the composition. In other embodiments, the flavorants or flavors have a synergistic effect with any sweeteners present. In other embodiments, any conventional, approved flavorants that do not materially affect the physical or chemical attributes of the active or of the resulting suspension can be added to the composition. In other embodiments, both natural and synthetic flavorants are used. In other embodiments, the flavorants are selected from natural and synthetic flavorants. In other embodiments, the flavorants include vanilla, strawberry, cherry, grape, lemon, lime, orange, peppermint, spearmint, cinnamon, or any combination thereof. In other embodiments, flavorants are added in amounts within the range of from about 0.005% to about 20% by weight of the composition. In other embodiments, flavorants are added in amounts within the range of from about 0.01% to about 5% by weight of the composition. In some embodiments, the compositions comprise cannabinoid extracts that contain a combination of at least two of the following: tetrahydrocannabinol (THC), cannabidiol (CBD), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), tetrahydrocannabinolic acid (THCa), cannabidiolic acid (CBDa), cannabinolic acid (CBNa), cannabichromenic acid (CBCa), and derivatives thereof. The cannabinoid may be natural or synthetic. In certain embodiments, the compositions comprise at least two cannabinoids that are in a 20:1 proportion by weight. In other embodiments, the compositions contain at least two cannabinoids that are in a 10:1 proportion by weight. In other embodiments, the compositions contain at least two cannabinoids that are in a 1:1 proportion by weight.

In certain embodiments, the cannabinoid extract is a mixture of cannabinoids and includes terpenes and/or flavonoids.

In other embodiments, the terpene comprises beta-myrcene, limonene, beta caryopyllene, caryopyllene oxide, terpineol, citronellol, linalool, humulene, beta-amyrin, cycloartenol, or a combination thereof. Any other suitable terpene can also be employed in accordance with the compositions and methods described herein.

The methods of making cannabinoid extract is well known in the art. The cannabis plants are grown, harvested, and the cannabinoids are extracted through, for example, a CO₂ extraction process.

In other embodiments, it may be advantageous to decarboxylate the inactive (carboxylic acid form) cannabinoids in the extracts and formulations.

In certain embodiments, the dosage form is in the form of a solid, inert core containing an isolated cannabis extract, in the form of a disc or cylinder, which increases, e.g., maximizes, the exposed surface area.

In other embodiments, the dosage form is in the form of a solid, inert core containing an isolated cannabis extract, that has the requisite structural strength to maintain its form during manufacture, packaging, insertion into the vaporization device, and over the shelf life of the product.

In certain embodiments, the dosage form is easy to manufacture and ensures complete vaporization through a vaporization device. In certain embodiments, “complete vaporization” means at least 75%, at least 80%, at least 85%, at least 90%, at least 95%, at least 98%, or at least 99% vaporization of the cannabinoid or cannabinoid extract.

In certain embodiments, the composition is for administration in a vaporized form. In other embodiments, the composition is for administration via a personal vaporizer.

In certain embodiments, the composition or dosage form is administered in a vaporized form. In certain embodiments, the composition or dosage form is administered via a personal vaporizer. As discussed above, personal vaporizers are devices that typically utilize a small battery-powered atomizer or heater to turn a substance into a vapor so that a subject can inhale the vapor. Personal vaporizers suitable for use with the compositions or dosage forms of the present invention include, but are not limited to, electronic cigarettes. A composition or dosage form can be loaded into the vaporizer directly or can be filled into a cartridge (i.e., into a composition storage compartment thereof) that can then be loaded into the vaporizer. The composition storage compartment (i.e., of the cartridge or of the vaporizer) is characterized by an internal volume. In certain embodiments, the personal vaporizer is an electronic cigarette.

The heater or heating element may be part of the vaporizer itself or may be part of the cartridge. In some embodiments, the personal vaporizer (e.g., the electronic cigarette) comprises one or more heating elements, a power supply (e.g., a battery), and a composition storage compartment. In other embodiments, the personal vaporizer (e.g., the electronic cigarette) comprises a power supply adapted for use with a cartridge comprising a composition storage compartment and one or more heating elements. In certain embodiments, the composition storage compartment comprises a vaporization chamber.

In certain embodiments, the cartridge is a container pod. The heater of the vaporizer may be controlled so as to maintain the temperature of the internal volume of the container pod within one or more ranges for a suitable period of time during the stage of vaporization. In some embodiments, the temperature within the internal volume of the container pod during vaporization may be maintained between 300° F. and 500° F., between 300° F. and 350° F., between 400° F. and 450° F., between 450° F. and 500° F., between 350° F. and 400° F., between 350° F. and 410° F., between 360° F. and 390° F., between 350° F. and 385° F., between 360° F. and 370° F., between 375° F. and 385° F. (e.g., approximately 378° F., approximately 380° F., approximately 382° F., etc.) for at least 5 seconds, at least 10 seconds, at least 15 seconds, at least 20 seconds, at least 30 seconds, at least 60 seconds, or for any other suitable period of time. It can be appreciated that other temperature ranges within the internal volume of the pod during vaporization may be maintained for an appropriate period of time.

In one aspect, provided herein are kits for administering a composition suitable for use in a personal vaporizer, the kit comprising: a dosage form as described herein; a personal vaporizer; and instructions for the use of said kit.

In other embodiments, provided are dosage forms that are processed and packaged for consistency, efficacy, and single-dose use.

In certain embodiments, the total weight of one cannabinoid is between 0.01 mg/dose and 300 mg/dose and the total weight of a second cannabinoid is between 0.01 mg/dose and 300 mg/dose.

In other embodiments, the total weight of one cannabinoid is between 0.01 mg/dose and 200 mg/dose and the total weight of a second cannabinoid is between 0.01 mg/dose and 200 mg/dose. In other embodiments, the total weight of one cannabinoid is between 0.01 mg/dose and 100 mg/dose and the total weight of a second cannabinoid is between 0.01 mg/dose and 100 mg/dose. In other embodiments, the total weight of one cannabinoid is between 0.01 mg/dose and 50 mg/dose and the total weight of a second cannabinoid is between 0.01 mg/dose and 50 mg/dose. In other embodiments, the total weight of one cannabinoid is between 0.01 mg/dose and 20 mg/dose and the total weight of a second cannabinoid is between 0.01 mg/dose and 20 mg/dose.

In certain embodiments, the unit dose comprises about 0.1-100 mg of at least one active ingredient, e.g., cannabinoid or cannabinoid extract. In other embodiments, the unit dose comprises about 0.1-10 mg of at least one active ingredient, e.g., cannabinoid or cannabinoid extract. In other embodiments, the unit dose comprises about 0.25-100 mg of at least one active ingredient, e.g., cannabinoid or cannabinoid extract. In other embodiments, the unit dose comprises about 0.25-0.5 mg of at least one active ingredient, e.g., cannabinoid or cannabinoid extract. In other embodiments, the unit dose comprises about 0.5-1 mg of at least one active ingredient, e.g., cannabinoid or cannabinoid extract. In other embodiments, the unit dose comprises about 1-2.5 mg of at least one active ingredient, e.g., cannabinoid or cannabinoid extract. In other embodiments, the unit dose comprises about 2.5-5 mg of at least one active ingredient, e.g., cannabinoid or cannabinoid extract. In other embodiments, the unit dose comprises about 5-7.5 mg of at least one active ingredient, e.g., cannabinoid or cannabinoid extract. In other embodiments, the unit dose comprises about 7.5-10 mg of at least one active ingredient, e.g., cannabinoid or cannabinoid extract. In other embodiments, the unit dose comprises about 10 mg of at least one active ingredient, e.g., cannabinoid or cannabinoid extract.

In certain embodiments, the unit dose comprises between 0.25 mg and 300 mg of total active ingredient comprising one or more cannabinoid or cannabinoid extract. In other embodiments, the unit dose comprises between 100 mg and 200 mg of total active ingredient comprising one or more cannabinoid or cannabinoid extract.

In certain embodiments, the composition comprises between 0.25 mg and 300 mg of total active ingredient comprising one or more cannabinoid or cannabinoid extract. In other embodiments, the composition comprises between 100 mg and 200 mg of total active ingredient comprising one or more cannabinoid or cannabinoid extract.

In certain embodiments, the composition includes a composition for daily administration. In certain embodiments, the therapeutic effect is maintained with one administration, twice daily. In other embodiments, the composition, the duration of each dose's effect is between four and six hours.

In certain embodiments, the composition is administered to a human subject. In other embodiments, the composition is administered to a human adult subject. In other embodiments, the composition is administered for single use. In other embodiments, the composition is in a single, measured unit.

In certain embodiments, the dosage form is configured for insertion into a chamber having a shape that has sufficient surface area for complete vaporization of the dosage form. For example, in certain embodiments, the composition is sized and shaped to be inserted into the vaporization chamber of an inhalation device. In other embodiments, the composition is inserted into a chamber having a shape that has sufficient surface area for complete vaporization of the dosage form. In certain embodiments, the inhalation device is a vaporizer, such as a personal vaporizer.

Examples of a disease or a disorder that can be treated by the invention include, but are not limited to, pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen- induced arthritis, atherosclerosis, and dystonia.

In some embodiments where the disorder is cancer, pain associated with cancer, nausea associated with chemotherapy, or a combination thereof, the composition described herein exerts reduced hallucinatory effects compared to smoking a cannabis containing cigarette or ingesting a cannabis containing foodstuff with the same amount of active ingredients.

In some embodiments, the cannabinoid is any member of a group of substances that are structurally related to tetrahydrocannabinol and that bind to a cannabinoid receptor such as CB1 or CB2 or both (‘THC’). The cannabinoid can be a naturally occurring compound (e.g., present in cannabis), a compound metabolized by a plant or an animal, or a synthetic derivative.

The cannabinoid may be included in its free form or in the form of a salt; an acid addition salt of an ester; an amide; an enantiomer; an isomer; a tautomer; a prodrug; a derivative of an active agent; different isomeric forms (for example, enantiomers and diastereoisomers), both in pure form and in admixture, including racemic mixtures and enol forms.

The cannabinoids are further meant to encompass natural cannabinoids, natural cannabinoids that have been purified or modified, and synthetically derived cannabinoids, for example, U.S. Patent Application Publication No. 2005/0266108, which is hereby incorporated by reference in its entirety, describes a method of purifying cannabinoids obtained from plant material.

The cannabinoids can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl) -1-(4-N-morpholinobutyryloxy)-delta-8-tetrahydrocannabinol hydrochloride, dexanabinol, nabilone, levonantradol, or N-(2-hydroxyethyl) hexadecanoamide. In some embodiments, the cannabinoids can be any of the non-psychotropic cannabinoid 3-dimethylnepty 11 carboxylic acid homologine 8, delta-8-tetrahydrocannabinol.

The above ingredients may be used by combining two or more members at an appropriate ratio.

All patents and literature references cited in the present specification are hereby incorporated by reference in their entirety.

The present invention will be specifically explained by way of examples, but these examples are not intended to limit the present invention.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Formulation Having Cannabinoid

Exemplary formulations described above are prepared using the methods described herein.

The formulation of the present invention is a solid, inert medium containing a cannabinoid or cannabinoid extract. The formulation is suitable for oral inhalation using a personal vaporizer.

It is to be understood that while the present example relates the composition having certain THCICBD ratios, other THCICBD ratios (e.g., 1:10, 10:1, 20:1, 1:20, etc.) are also encompassed by the invention and can be easily prepared using methods known in the art.

Having described embodiments of the present invention, it is to be understood that the present invention is not limited to the above-mentioned embodiments and that various changes and modifications can be affected by one skilled in the art without departing from the spirit or scope of the present invention as defined in the appended claims.

We claim:

1. A dosage form suitable for use in an inhalation device, comprising a solid, inert medium containing a cannabinoid or cannabinoid extract.

2. The dosage form of claim 1, wherein the cannabinoid or cannabinoid extract is absorbed onto the solid medium.

3. The dosage form of claim 1 or 2, wherein said medium is a core.

4. The dosage form of any one of claims 1-3, wherein said medium comprises organic and inorganic compounds.

5. The dosage form of any one of claims 1-4, further comprising a pharmaceutically acceptable solvent into which the cannabinoid is solvated and a pharmaceutically acceptable adsorbent onto which the solvated cannabinoid is adsorbed.

6. The dosage form of claim 5, wherein said solvent is a vegetable oil, hydrogenated vegetable oils, limonene, a terpene, an essential oil, polyethylene glycols (PEG), propylene glycol, ethanol, substituted polyethylene glycols, glycerin, mineral oil, oleic acid, fatty acid esters, benzyl alcohol, an alcohol, or any combinations thereof.

7. The dosage form of claim 5, wherein said solvent is hydrogenated vegetable oils, ethanol, or a mixture thereof.

8. The dosage form of claim 5, where said adsorbent is silica, microcrystalline cellulose, cellulose, silicified microcrystalline cellulose, starch, pregelatinized starch, dicalcium phosphate, or a mixture thereof.

9. The dosage form of claim 5, where said adsorbent comprises silica.

10. The dosage form of any one of claims 1-9, further comprising one or more other pharmaceutically acceptable excipients.

11. The dosage form of claim 10, wherein said one or more pharmaceutically acceptable excipients are selected from diluents, lubricants, granulating aids, colorants, flavorants, surfactants, pH adjusters, anti-adherents, glidants, and any combination thereof.

12. The dosage form of claim 11, wherein said lubricants are tableting lubricants and are selected from magnesium stearate, stearic acid, palmitic acid, calcium stearate, talc, polyethylene glycol, colloidal silicon dioxide, sodium stearyl fumarate, carnauba wax, and mixtures thereof.

13. The dosage form of claim 11 or 12, wherein said tableting lubricants are present in an amount of from about 0.2% to about 8% by weight.

14. The dosage form of claim 11 or 12, wherein said tableting lubricants are present in an amount of from about 0.5% to about 2% by weight.

15. The dosage form of any one of claims 11-14, wherein said flavorants are natural and synthetic flavorants.

16. The dosage form of claim 15, wherein said flavorants are vanilla, strawberry, cherry, grape, lemon, lime, orange, peppermint, spearmint, cinnamon, or any combination thereof.

17. The dosage form of any one of claims 11-16, wherein said flavorants are present in an amount of from about 0.005% to about 20% by weight.

18. The dosage form of any one of claims 11-16, wherein said flavorants are present in an amount of from about 0.01% to about 5% by weight.

19. The dosage form of any one of claims 1-18, wherein the dosage form is a pellet.

20. The dosage form of any one of claims 1-19, wherein the dosage form is temperature dependent.

21. The dosage form of any one of claims 1-20, wherein the dosage form is capable of accurate dosing via an inhalation device.

22. The dosage form of any one of claims 1-21, wherein the dosage form is for single use.

23. The dosage form of any one of claims 1-22, wherein the dosage form is for daily administration to a human subject.

24. The dosage form of any one of claims 1-23, wherein therapeutic effect is maintained with one administration, twice daily.

25. The dosage form of any one of claims 1-24, wherein the duration of the therapeutic effect of a single dose is between four and six hours.

26. The dosage form of any one of claims 1-25, wherein said inhalation device is a personal vaporizer.

27. The dosage form of any one of claims 1-26, wherein the cannabinoid or cannabinoid extract in the dosage form is for administration in a vaporized form.

28. The dosage form of any one of claims 1-27, wherein the dosage form is sized and shaped to be inserted into a vaporization chamber of said inhalation device.

29. The dosage form of any one of claims 1-28, wherein the dosage form is configured for insertion into a vaporization chamber having a shape that has sufficient surface area for complete vaporization.

30. The dosage form of any one of claims 1-29, wherein the dosage form is in the form of a disc or cylinder.

31. The dosage form of any one of claims 1-30, wherein the dosage form is in a single, measured unit.

32. The dosage form of any one of claims 1-31, wherein the cannabinoid or canninboid extract is selected from tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), and cannabichromene (CBC).

33. The dosage form of any one of claims 1-32, wherein the cannabinoid or cannabinoid extract comprises at least two cannabinoids.

34. The dosage form of claim 33, wherein the two cannabinoids are selected from tetrahydrocannabinol (THC), cannabidiol (CBD), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), and derivatives thereof.

35. The dosage form of claim 33 or 34, wherein the two cannabinoids are THC and CBD.

36. The dosage form of claim 33 or 34, wherein the two cannabinoids are THCa and CBDa.

37. The dosage form of any one of claims 33-36, wherein the two cannabinoids are in a 1:1 proportion by weight.

38. The dosage form of any one of claims 33-36, wherein the two cannabinoids are in a 10:1 proportion by weight.

39. The dosage form of any one of claims 33-36, wherein the two cannabinoids are in a 20:1 proportion by weight.

40. The dosage form of any one of claims 1-39, wherein the total amount of the cannabinoid or cannabinoid extract is between about 0.1 mg and about 10 mg.

41. The dosage form of any one of claims 1-39, wherein the cannabinoid or cannabinoid extract is THC and is present in an amount ranging from about 0.1 mg to about 10 mg.

42. The dosage form of any one of claims 1-39, wherein the cannabinoid or cannabinoid extract is CBD and is present in an amount ranging from about 0.1 mg to about 10 mg.

43. A method for treating a disease or a disorder in a subject, comprising administering to the subject a therapeutically effective amount of the dosage form of any one of claims 1-42.

44. The method of claim 43, wherein the disease or the disorder is selected from pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis, and dystonia.
